(19) United States
(12) Patent Application Publication
(10) Pub. No.: US 2007/0207953 A1
(43) Pub. Date: Sep. 6, 2007

(54) Host Defense Factor X (HDFX)

(76) Inventor: Burton M. Altura, Jersey City, NJ (US)

Correspondence Address: SCULLY, SCOTT, MURPHY & PRESSER, P.C., 400 GARDEN CITY PLAZA, SUITE 300, GARDEN CITY, NY, 11530, US

(21) Appl. No.: 10/564,946

(22) PCT Filed: Jul. 16, 2004
(86) PCT No.: PCT/US04/22917

§ 371 Date: Mar. 12, 2007

Related U.S. Application Data
(60) Provisional application No. 60/487,668, filed on Jul. 16, 2003.

(51) Int. Cl. A61K 38/16; C07K 14/47

(52) U.S. Cl. 514/12; 530/350

(57) ABSTRACT

The present invention relates to compositions and methods useful in medical prophylaxis and treatment of a mammalian subject before and after exposure of the subject to potentially lethal pathogens and other insults. More specifically, the present invention provides an isolated a natural host defense factor, or “HDFX”, which enhances an animal subject's ability to withstand infection by any pathogen and a variety of insults. Pharmaceutical compositions containing HDFX and therapeutic methods involving administration of HDFX are also provided.

### FIELD OF THE INVENTION

The present invention relates to compositions and methods useful in medical prophylaxis and treatment of a subject before and after exposure of the subject to potentially lethal pathogens and other insults. More specifically, the present invention provides an isolated natural host defense factor, or “HDFX”, which enhances a subject's ability to withstand infection by any pathogen and a variety of insults. Pharmaceutical compositions containing HDFX and therapeutic methods involving administration of HDFX are also provided.

### BACKGROUND OF THE INVENTION

The terrorist attack on Sep. 11, 2001 and the Anthrax scare that followed have focused attention on chemical and biological warfare by terrorist groups and rogue states. Clearly there is a need for new vaccines against anthrax and a host of other lethal pathogens such as smallpox, plague, botulism, viral hemorrhagic fever, and tularemia, among many others. However, it is impossible to prepare and dispense vaccines for every member of a population and inoculate everyone against every potential biological agent or vector. In addition, vaccines are often associated with various side effects, ranging from brain damage to death. Since the human body has natural resistance to disease, it would be desirable to develop medical prophylaxis and treatment for use before and after exposure to potentially lethal pathogens, which can boost the body's natural resistance to disease.

### SUMMARY OF THE INVENTION

The present inventor has unexpectedly identified a natural host defense factor, or “HDFX”, which can super-boost an animal subject's immune system to fight against or recover from a disease or condition caused by any pathogen, and to withstand a variety of insults.

In one embodiment, the present invention provides an isolated and partially purified HDFX.

In another embodiment, the present invention provides a pharmaceutical composition suitable for administration to a subject, which contains isolated or partially purified HDFX and a pharmaceutically acceptable carrier. A “subject” as used herein includes any mammalian subject, such as primate (e.g., human), mice, rats, dogs, cats, cattle, sheep and pigs, for example. Preferred subjects include companion animals. A companion animal refers to any non-human animal considered to be a pet, including but not limited to, dogs, cats, rabbits, monkeys, among others. An especially preferred subject is a human subject.

In still another embodiment, the present invention provides a method of enhancing or potentiating the ability of a subject to fight against or recover from a disease or condition caused by a pathogen by administering an effective amount of HDFX to the subject either before or after exposure to the pathogen.

The term “pathogen” as used herein includes bacterial (such as anthrax), fungal, viral (such as HIV and hepatitis virus), parasite (protozoa, worms, cryptosporidia, among others), and prion or prion-like molecules.

The term “disease or condition” includes, but is not limited to, diseases caused by anthrax, smallpox, plague, botulism, hemorrhagic fever, HIV, infection caused by a hepatitis virus (e.g., hepatitis B), and the virus resulting in severe acute respiratory syndroe (SARS), and new emerging diseases among others.

In a further embodiment, the present invention provides a method of enhancing the ability of an animal subject to sustain and survive an insult by administering an effective amount of HDFX to the animal prior to and/or after the insult.

The term “insult” includes, e.g., trauma, hemorrhage, burns, select poisons, hemorrahagic strokes, ischemic strokes, sepsis, multiple organ failure, battle wounds, tumor invasion, neurosurgical procedures, major surgery, chemicals used in biological warfare, and prolonged generalized systemic stress (e.g., exposure to high altitude and space flight).

### BRIEF DESCRIPTION OF THE DRAWINGS

FIG. 1. Res phagocytic function is compromised early after whole-body trauma, hemorrhage and bowel ischemia.

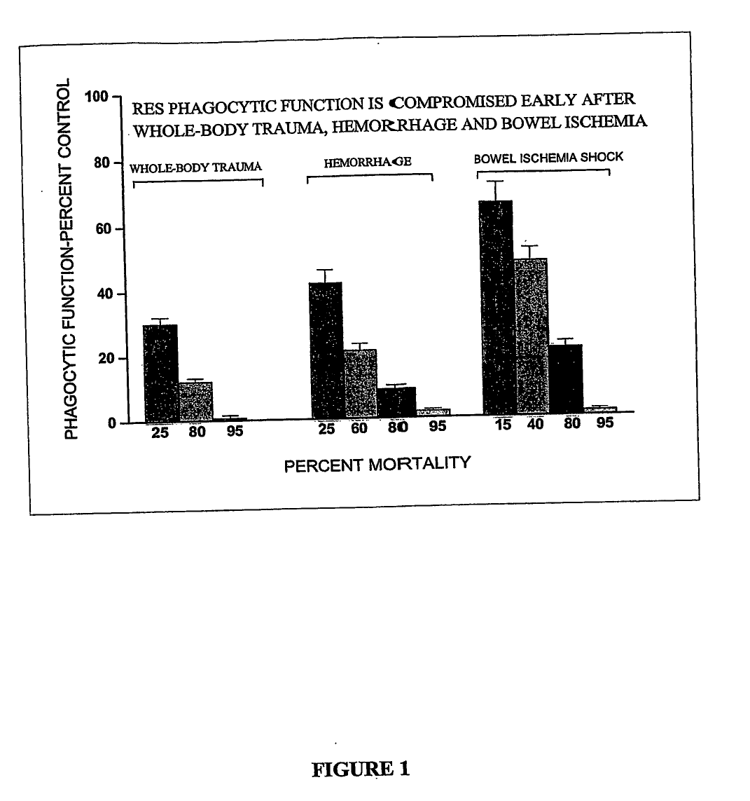

FIG. 2. HDFX treatment prevents death induced by various lethal bacteria in rats.

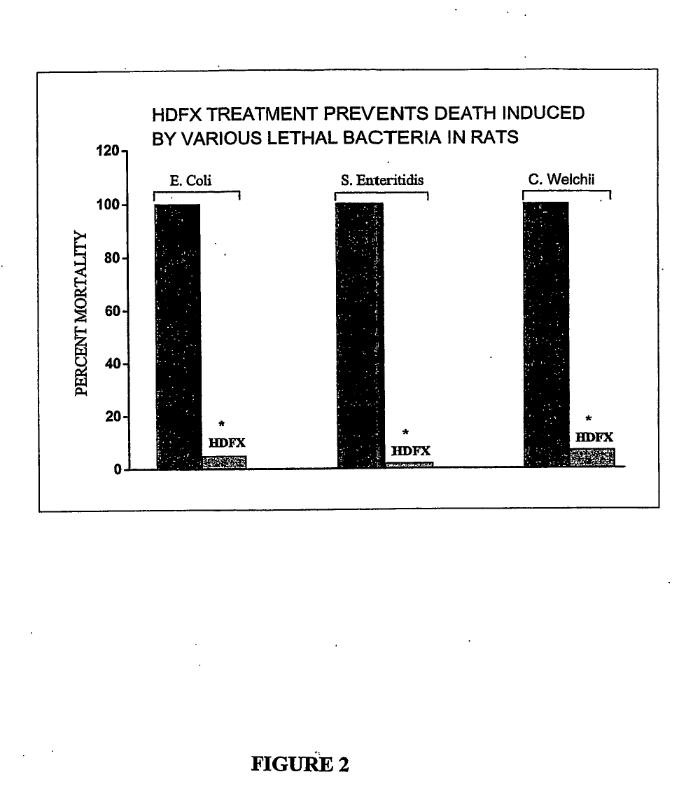

FIG. 3. HDFX treatment prevents loss of cytokines in lymphocytes from rats subjected to hemorrhage or trauma.

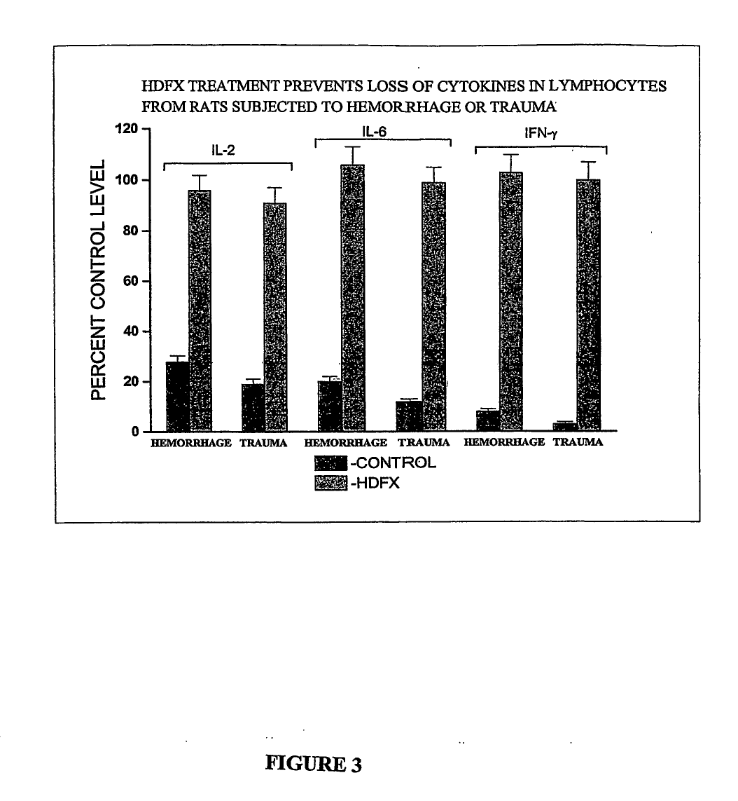

### DETAILED DESCRIPTION OF THE INVENTION

The present inventor has surprisingly found that biological response modifiers can act as stimulants for the production and release of naturally-occurring host defense factors, and that these naturally-occurring host defense factors can increase human tolerance to a variety of insults such as lethal pathogens, circulatory shock, trauma, burns, strokes, wounding and sepsis.

More specifically, the present inventor has identified a natural host defense factor, or “HDFX”, which can super-boost a subject's immune system to withstand and recover from a disease caused by any pathogen (bacterial, fungal or viral), and to withstand a variety of insults such as trauma, hemorrhage, ischemic strokes, sepsis, burns, multiple organ failure, battle wound, major surgery.

As used herein, the term “subject” means any mammalian subject, including primate (e.g., human), mice, rats, dogs, cats, cattle, sheep and pigs, for example. Preferred subjects include companion animals. A companion animal refers to any non-human animal considered to be a pet, including but not limited to, dogs, cats, rabbits, monkeys, among others. An especially preferred subject is a human subject.

In one embodiment, the present invention provides an isolated and partially purified HDFX.

The isolation of murine and rat HDFX is described herein below. Those skilled in the art can isolate HDFX from another animal subject using procedures comparable to the procedure for isolating murine and rat HDFX.

According to the present invention, there are two sources from which a murine or rat HDFX can be isolated. HDFX can be extracted from mice or rats which have survived repeated insults (“survival mice” or “survival rats”), such as hemorrhage, bowel ischemia, trauma and septic shock. Alternatively, HDFX can be extracted from mice or rats which have been properly stimulated.

To isolate HDFX, pooled plasma from stimulated or survival mice or rats is brought to about pH 5.5 by addition of 1N HCl, boiled for 10 minutes and filtered. The material is stored in stoppered vials and frozen until use. Alternatively, appropriate organ-tissues, such as bone marrow, spleen, lymphoid nodes, or liver, among others, are excised and homogenized at 4° C. Acidic saline (pH 5.5) is added during homogenization. The sample is then boiled for 10 minutes and filtered. The extracts are stored frozen (−10 to −20° C.). Such partially purified HDFX can be further purified using any of protein or glycoprotein purification procedures available to those skilled in the art.

According to the present invention, HDFX is water soluble, resistant to about pH 5.5 and to boiling for 10 minutes. It is stable at 4° C. for at least one week and at −20° C. for several weeks. It is nondialyzable and can be lyophilized. It is believed that HDFX contains a glycoprotein component, and may be a single molecule or a combination of more than one molecule.

The activities of HDFX manifest in several aspects. In the first instance, endogenous stimulation of HDFX in a mammalian subject enhances survival of such subject after hemorrhage, trauma, burns and sepsis. In addition, HDFX treatment stimulates res phagocytic function in normal mammalian subjects. Importantly, res phagocytic function is typically compromised in mammals early after whole-body trauma, hemorrhage, burns and bowel ischemia. Furthermore, treatment of a subject with HDFX prevents death of such subject induced by various lethal bacteria. Moreover, HDFX treatment prevents loss of cytokines in lymphocytes from mammals subjected to hemorrhage or trauma. These activities of HDFX are further illustrated in the Examples set forth hereinbelow.

The identification and isolation of HDFX, achieved by the present invention, allows development of a pharmaceutical composition that boosts a subject's immune function and kills most, if not all, lethal bacteria and viruses, natural or man-made.

Accordingly, in another embodiment, the present invention provides a pharmaceutical composition suitable for administration to a subject, which contains isolated or partially purified HDFX and a pharmaceutically acceptable carrier.

As used herein, a pharmaceutically acceptable carrier includes any and all solvents, dispersion media, isotonic agents, a delivery substance or vehicle (e.g., effervescent tablets), and the like. Except insofar as any conventional media, agent, diluent or carrier is detrimental to the recipient or to the activity of HDFX, its use in the pharmaceutical composition of the present invention is appropriate. The carrier can be liquid, semi-solid, e.g. pastes, or solid carriers. Examples of carriers include oils, water, saline solutions, alcohol, sugar, gel, lipids, liposomes, resins, porous matrices, binders, fillers, coatings, preservatives and the like, or combinations thereof.

In accordance with the present invention, HDFX can be combined with the carrier in any convenient and practical manner, e.g., by admixture, solution, suspension, emulsification, encapsulation, absorption and the like, and can be made in formulations such as tablets including effervescent tablets, capsules, powder, syrup, suspensions that are suitable for injection, implantation, inhalation, ingestion or the like.

In still another embodiment, the present invention provides a method of enhancing or potentiating the ability of a mammal to fight against or recover from a disease or condition caused by exposure to a pathogen, by administering an effective amount of HDFX to the subject either before or after the exposure to the pathogen. Preferably, HDFX used in the administration is provided in a pharmaceutically acceptable carrier, which is described hereinabove.

Without intending to be bound by any particular theory, it is believed that HDFX can potentiate a mammal's ability to fight against or recover from infection by a pathogen through enhancing or stimulating the production and/or function of cells of the mammal's immune system, including macrophages, NK cells, T cells and B cells. Alternatively, HDFX acts to stimulate the production of cytokines, such as IL-2, IL-6, INF-γ, and TNF-α. HDFX may also act to adapt the body to inflammatory conditions such as blood loss, burns, trauma, wounding or combined injuries.

The protective effects of HDFX are not limited to any particular pathogen, and are applicable to bacterial (such as anthrax), fungal, viral (such as HIV and hepatitis virus), prion-like pathogens, ricketsial organisms and mycoplasms.

The term “disease or condition” includes, but is not limited to, disease caused by anthrax, smallpox, plague, botulism, hemorrhagic fever, tellaremia, severe acute respiratory syndrome caused by the SARS virus, HIV, infection caused by a hepatitis virus (e.g., hepatitis B), among others.

The methods of the present invention are effective for the prevention of hospital (nosocomial) infections. Currently, there are a million American's infected by hospitals per year. Of these, approximately 100,000 of them die each year. HDFX prophylaxis and treatment of each patient admitted to hospitals should reduce the number of these nosocomial infections by 70-90%.

In a further embodiment, the present invention provides a method of enhancing the ability of an animal subject to sustain and survive an insult by administering an effective amount of HDFX to the animal prior to and/or after the insult. The insult includes, e.g., trauma, hemorrhage, ischemic strokes, sepsis, burns, multiple organ failure, battle wounds, and major surgery. Preferably, HDFX used in the administration is provided in a pharmaceutically acceptable carrier, which is described hereinabove.

Without intending to be bound by any particular theory, it is believed that HDFX can potentiate a subject's ability to sustain and survive an insult by way of enhancing or stimulating the production and/or function of cells of the subject's immune system, including macrophages, NK cells, T cells and B cell. Alternatively, HDFX acts to stimulate the production of cytokines, such as IL-2, IL-6, INF-γ, and TNF-α. HDFX may also act to adapt the body to inflammatory conditions such as blood loss, trauma, burns, or combined injuries.

Therefore, HDFX can be used to enhance survival rates and shorten the hospital stays of patients undergoing open-heart, cancer, and neurosurgical procedures, of victims, and of armed forces personal subjected to potential battlefield insults and injuries and chemical weapons.

By virtue of HDFX's macrophage-stimulating activities, HDFX can also be used to increase the activities of infection-fighting cells in cancer patients who have bone marrow transplants, and allow oncologists to decrease chemotherapy doses prescribed for these cancer patients.

The present invention is further illustrated by, but not limited to, the following examples.

### EXAMPLES

TABLE 1
Effects of Endogenous Stimulation of HDFX on Survival
After Hemorrhage, Trauma and Sepsis in Rats

No. Died/Total No.

| TREATMENT | Hemorrhage | Trauma | Sepsis |
|---|---|---|---|
| A. Stimulant 1 | | | |
| Controls | 10/24 | 16/22 | 23/24 |
| | (42)* (73) | (96) | |
| Treatment | 1/24 | 2/22 | 2/24 |
| | (4)^{†} | (9)^{†} | (8)^{†} |
| B. Stimulant 2 | | | |
| Controls | 10/26 | 12/19 | 22/23 |
| | (38) | (63) | (96) |
| Treatment | 3/26 | 3/19 | 5/23 |
| | (12)^{†} | (16)^{†} | (22)^{†} |
| C. Stimulant 3 | | | |
| Controls | 10/18 | 16/20 | 18/20 |
| | (80) | (80) | (90) |
| Treatment | 4/18 | 5/20 | 8/20 |
| | (22)^{†} | (40)^{†} | (40)^{†} |

*Percent Mortality.
^{†}P < 0.01.

TABLE 2
HDFX Treatment Stimulates Res Phagocytic
Function in Normal, Naïve Rats

| Group | N | Phagocytic Index |
|---|---|---|
| Control | 20 | 0.48 ± 0.004 |
| HDFX | 26 | 0.135 ± 0.008* |

*P < 0.01

TABLE 3
Effects of Hemorrhage and Trauma W/Without HDFX Treatment
on Res Phagocytic Function

| Group | N | K-value |
|---|---|---|
| Controls | 25 | 0.050 ± 0.005 |
| A.Hemorrhage | | |
| Controls | 16 | 0.008 ± 0.002* |
| HDX | 12 | 0.047 ± 0.006 |
| B. Trauma | | |
| Controls | 20 | 0.010 ± 0.004* |
| HDFX | 14 | 0.046 ± 0.005 |

*P < 0.01

TABLE 4
Mortality in Rats Subjected to Hemorrhage, Trauma and Sepsis
With or Without HDFX Treatment

| TREATMENT | N | No. Died/Total No. | % MORTALITY |
|---|---|---|---|
| A. Hemorrhage | | | |
| Controls | 22 | 10/22 | 45 |
| Treatment | 22 | 1/22 | |
| B. Trauma | | | |
| Controls | 24 | 20/24 | 83 |
| Treatment | 24 | 1/24 | |
| C. Sepsis | | | |
| Controls | 18 | 12/18 | 67 |
| Treatment | 18 | 1/18 | |

^{†}P < 0.01

TABLE 5
Effects of Hemorrhage and Trauma on Soluble (Cytokine) Mediators
in Rat Lymphocytes

| | | U/ml | |
|---|---|---|---|
| Mediators | Controls-shams | Hemorrhage | Trauma |
| IL-2 | 402 ± 75 | 110 ± 20* | 75 ± 15* |
| IL-6 | 1.7 ± 0.3 | 0.35 ± 0.12* | 0.22 ± 0.08* |
| IFN-γ | 7.5 ± 0.3 | 0.65 ± 0.11* | 0.21 ± 0.05* |
| TGF-β | 14.2 ± 2.2 | 29.5 ± 4.3* | 37.8 ± 4.7* |
| (ng/ml) | | | |

Values are mean ± S.E.M.
*P < 0.01.

What is claimed is:

1. An isolated natural host defense factor (HDFX).

2. The isolated HDFX of claim 1, wherein said HDFX is of a mammalian origin.

3. The isolated HDFX of claim 2, wherein said HDFX is murine or rat HDFX.

4. The isolated HDFX of claim 1, wherein said HDFX is water soluble, and is resistant to pH 5.5 and to boiling for 10 minutes.

5. A pharmaceutical composition comprising the isolated HDFX of any one of claims 1-3, and a pharmaceutically acceptable carrier.

6. A method of enhancing the ability of a subject to fight against or recover from a disease caused by a pathogen, comprising administering an effective amount of HDFX to the animal before or after exposure to the pathogen.

7. The method of claim 6, wherein said subject is a mammal.

8. The method of claim 7, wherein said mammal is human.

9. The method of claim 6, wherein said subject is a companion animal.

10. The method of claim 6, wherein said pathogen is a bacterial, fungal, mycoplasm, ricketsial, parasite, prion-like or viral pathogen.

11. The method of claim 10, wherein said pathogen is selected from anthrax, HIV or a hepatitis virus.

12. The method of claim 6, wherein said disease is selected from smallpox, plague, botulism, hemorrhagic fever, AIDS, fever, severe acute respiratory syndrome, a disease caused by anthrax, or a disease caused by hepatitis B virus, or new evolving diseases.

13. A method of enhancing the ability of a subject to sustain and survive an insult by administering an effective amount of HDFX to the subject prior to or after the insult.

14. The method of claim 13, wherein said insult is selected from the group consisting of trauma, hemorrhage, ischemic strokes, sepsis, burns, multiple organ failure, battle wounds, and major surgery.
